(19) United States
(12) Patent Application Publication
(10) Pub. No.: US 2009/0149847 A1
(43) Pub. Date: Jun. 11, 2009

(54) SYSTEMS AND METHODS FOR PUNCTURE CLOSURE

(75) Inventors: Amnon Yadin, Kfar Vitkin (IL); Noam Mizrahi, Kfar Vitkin (IL); Aharon Cohen, Zichron Yaakov (IL)

Correspondence Address: PATTON BOGGS LLP, 8484 WESTPARK DRIVE, SUITE 900, MCLEAN, VA, 22102, US

(73) Assignee: CardioDex Ltd., Cesarea Business Park (IL)

(21) Appl. No.: 12/245,569

(22) Filed: Oct. 3, 2008

Related U.S. Application Data
(60) Provisional application No. 60/960,604, filed on Oct. 5, 2007.
(60) Provisional application No. 61/006,926, filed on Feb. 6, 2008.

(51) Int. Cl. A61B 17/12; A61M 29/00; A61B 18/04

(52) U.S. Cl. 606/27; 606/213; 606/191

(57) ABSTRACT

Systems, apparatus and methods are provided for closing a puncture in a vessel wall may have various combinations of an anchoring device, a gripping device and a heating element for sealing a puncture within a vessel wall. An anchoring device may be a balloon, floating anchor, an expansible cage and/or a wire operated T-anchor. A gripping device may hold and/or twist the vessel wall to secure the vessel wall in place during heating. A heating element may provide heat to target tissue held by the gripping device.

### CROSS-REFERENCE TO RELATED APPLICATIONS

This application claims priority to U.S. Provisional Patent Application No. 60/960,604, filed Oct. 5, 2007 and U.S. Provisional Patent Application No. 61/006,926, filed Feb. 6, 2008, the contents of which are incorporated by reference herein in their entireties. This application incorporates by reference in their entireties U.S. Provisional Patent Application No. 60/935,484, filed Aug. 15, 2007, U.S. Non-Provisional patent application Ser. No. 12/192,911, filed Aug. 15, 2008 and PCI/US2008/073402, filed Aug. 15, 2008.

### FIELD OF THE INVENTION

The invention relates generally to methods and apparatus for treating vasculatures, and, more particularly, to methods and apparatus for post-catheterization closure of punctures within vessel walls.

### BACKGROUND OF THE INVENTION

Various techniques are known for arterial catheterization. Following arterial catheterization, it is necessary to promote hemostasis quickly and without undue hardship for the patient. Current systems may be either inefficient or painful for a patient.

Generally, needs exist for improved apparatus and methods for treating vasculatures. More specifically, needs exist for improved apparatus and methods for efficiently and effectively sealing a puncture in a vasculature.

### SUMMARY OF THE INVENTION

Embodiments of the present invention solve many of the problems and/or overcome many of the drawbacks and disadvantages of the prior art by providing an apparatus and method for sealing punctures in a wall of a vasculature.

In particular, embodiments of the invention may accomplish this with an apparatus including an anchor shaft, an anchoring device on a distal end of an anchor shaft wherein the anchoring device is deployed within a vessel lumen and withdrawn into contact with an inner surface of a vessel wall, a gripping shaft for passing over the anchor shaft, a gripping tip on a distal end of the gripping shaft for contacting an outer surface of the vessel wall and capturing tissue during rotation of the gripping shaft, and a heating element for passing through the gripping shaft into contact with the captured tissue.

Embodiments of the present invention may further include an introducer wherein the gripping shaft passes through the introducer and/or a sheath for covering the gripping shaft during passing over the anchor shaft.

The gripping tip may include extensions for gripping tissue where the extensions may be on an inward-facing, sloped wall at the distal end of the gripping shaft.

Embodiments of the present invention may also include a method of operating a system for closing a puncture in a vessel wall including providing a system for closing a puncture in a vessel wall including an anchor shaft, an anchoring device on a distal end of an anchor shaft, and a gripping shaft for passing over the anchor shaft, a gripping tip on a distal end of the gripping shaft, and a heating element, inserting the distal end of the anchor shaft into a vessel lumen, deploying the anchoring device within the vessel lumen, withdrawing the anchoring device into contact with an inside surface of a vessel wall, inserting the gripping shaft near the vessel wall, contacting the gripping tip with an outside surface of the vessel wall opposite the anchoring device, rotating the gripping shaft for capturing tissue, withdrawing the anchoring device through the gripping shaft, contacting the captured tissue with the heating element, and activating the heating element.

Embodiments of the present invention may also be a system for closing a puncture in a vessel wall including an anchor shaft, an anchoring device on a distal end of the anchor shaft wherein the anchoring device is deployed within a vessel lumen and withdrawn into contact with an inner surface of a vessel wall, a second shaft, one or more prongs on a distal end of the second shaft for capturing tissue at an outer surface of the vessel wall opposite the anchor device, and a heating element within the second shaft for contacting the captured tissue.

Embodiments of the present invention may also include an expansion device within the second shaft for releasing the captured tissue, wherein the expansion device may be a balloon. The one or more prongs may be inward facing and/or disposed in two or more concentric rows wherein the one or more prongs may be angled in opposite directions in adjacent concentric rows.

In embodiments of the present invention, the anchoring device may be selected from the group consisting of a balloon, a T-anchor, an expansible cage, a floating anchor, and combinations thereof, or an inverting cup deployed in a first position, inverted, and withdrawn to capture tissue on the one or more prongs. The second shaft further may include a proximate internal tube and a distal external tube, wherein the internal tube rotates relative to the external tube. A twist control pin may be included for preventing over-rotation of the internal tube. A dilator may be included with an expansible section, wherein the dilator at least partially covers the distal external tube, wherein the expansible section opens upon movement of the external tube toward a vessel wall.

Embodiments of the present invention may include a method of operating a system for closing a puncture in a vessel wall including providing a system for closing a puncture in a vessel wall including an anchor shaft, an anchoring device on a distal end of the anchor shaft, a second shaft, one or more prongs on a distal end of the second shaft, and a heating element within the second shaft, inserting the distal end of the anchor shaft into a vessel lumen, deploying the anchoring device within the vessel lumen, withdrawing the anchoring device into contact with an inner surface of a vessel wall, inserting the second shaft over the anchor shaft proximate to an outer surface of the vessel wall opposite the anchoring device, capturing tissue on the one or more prongs, and applying the heating element to the captured tissue.

Embodiments of the present invention may further include withdrawing the anchoring device through the second shaft to capture the tissue on the one or more prongs, deploying an expansion device within the second shaft to release the captured tissue and undeploying the expansion device, withdrawing the anchoring device through the second shaft after capturing the tissue on the one or more prongs by rotating the one or more prongs, and/or providing a dilator with an expansible section and opening the expansible section by moving the external tube toward a vessel wall.

Additional features, advantages, and embodiments of the invention are set forth or apparent from consideration of the following detailed description, drawings and claims. Moreover, it is to be understood that both the foregoing summary of the invention and the following detailed description are exemplary and intended to provide further explanation without limiting the scope of the invention as claimed.

### BRIEF DESCRIPTION OF THE INVENTION

The accompanying drawings, which are included to provide a further understanding of the invention and are incorporated in and constitute a part of this specification, illustrate preferred embodiments of the invention and together with the detailed description serve to explain the principles of the invention. In the drawings:

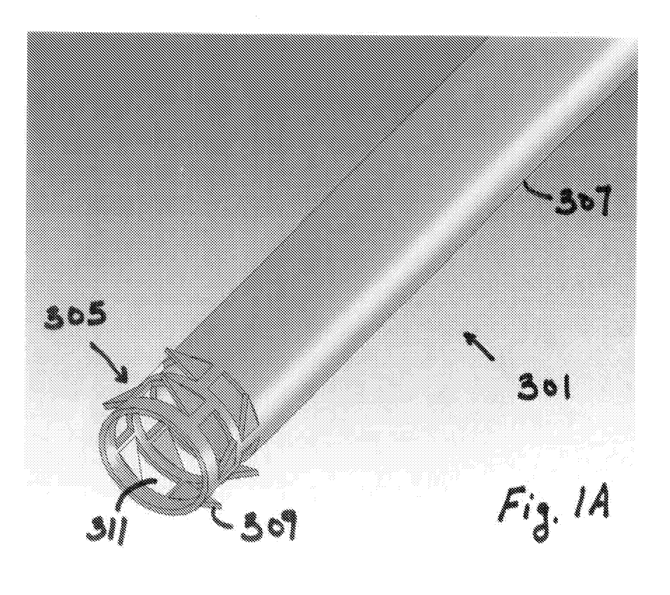

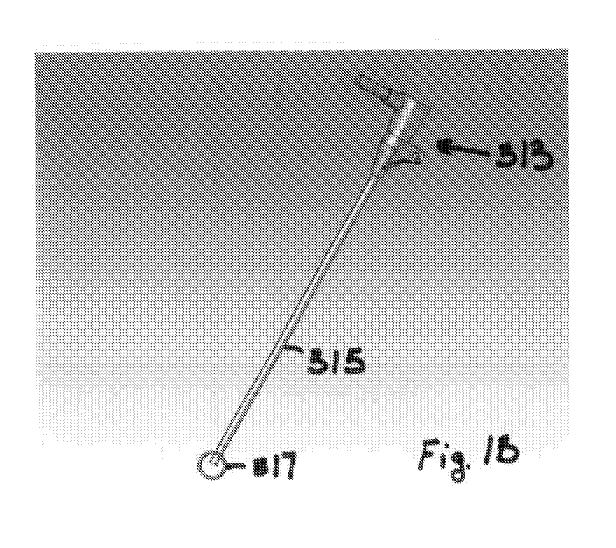

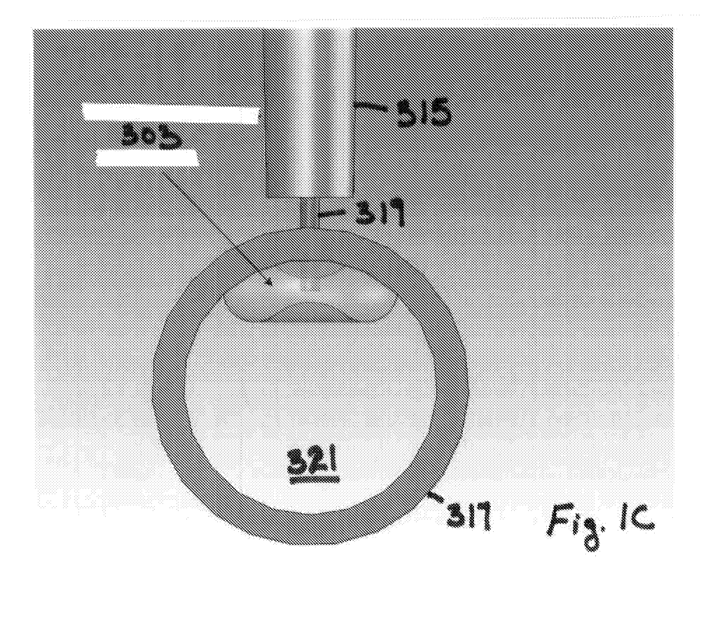

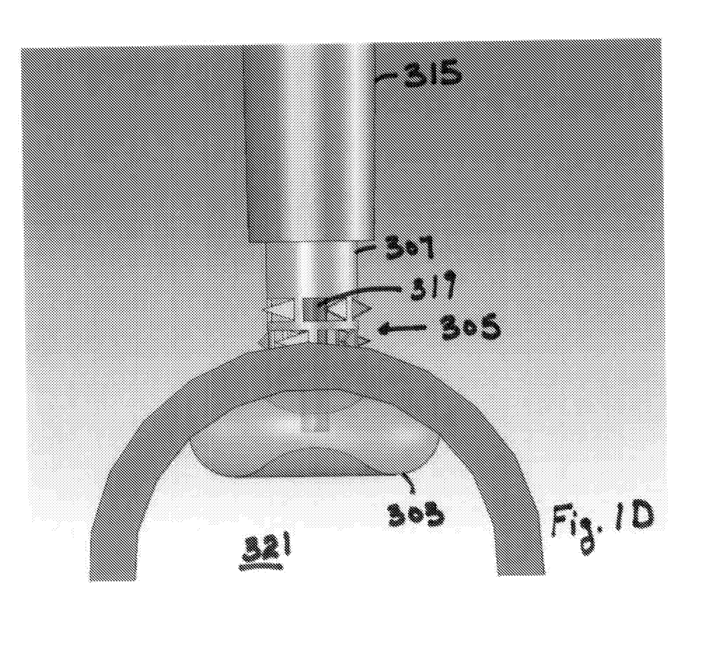

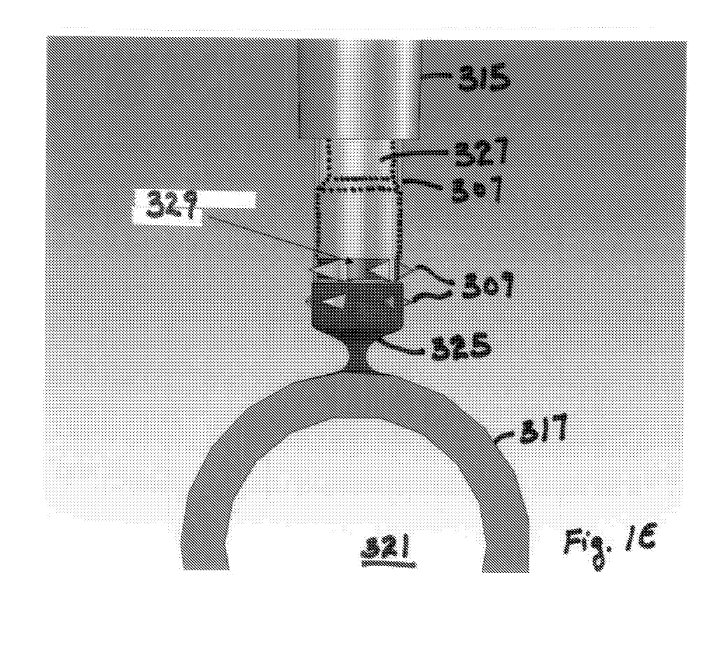

FIGS. 1A-1F illustrate operation of a system for closing a puncture in a vessel wall with an anchoring device and a wall grip end.

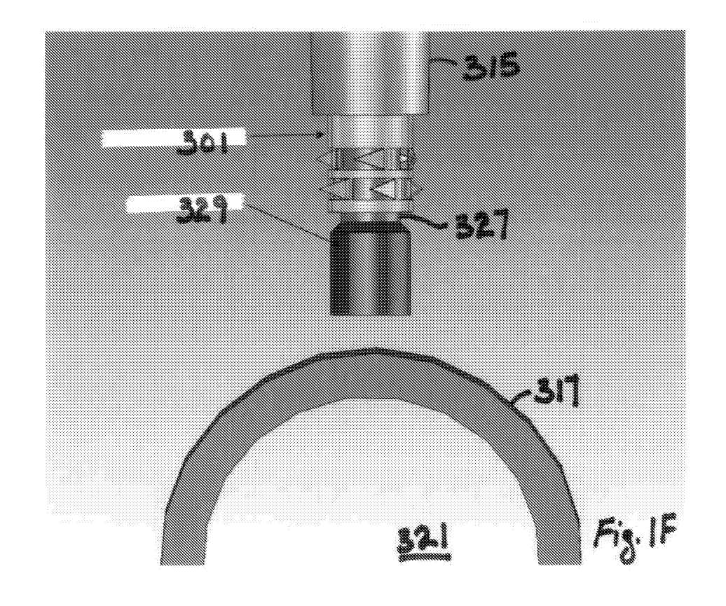

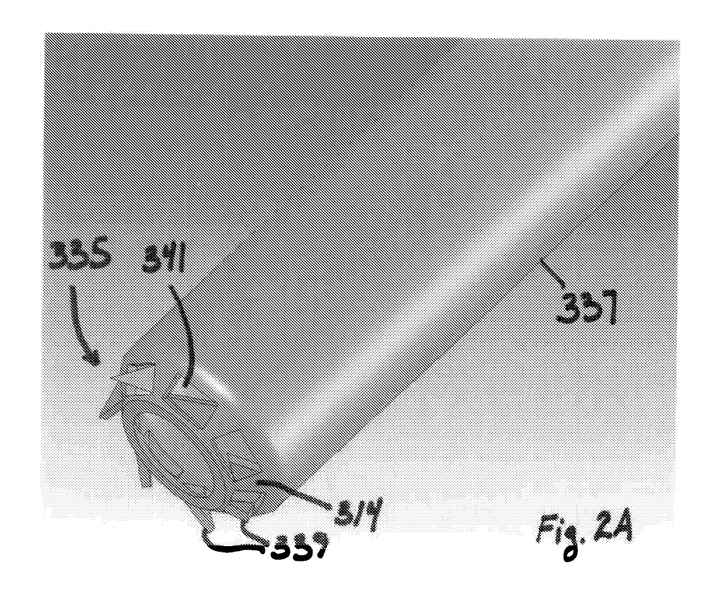

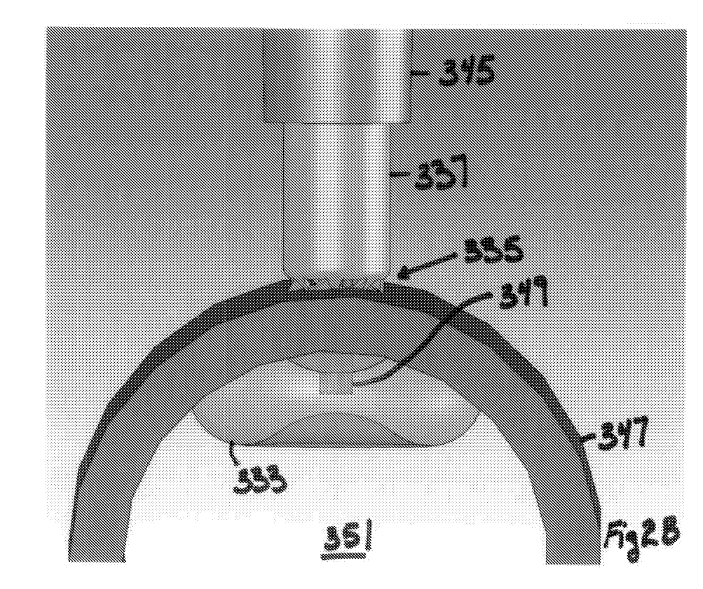

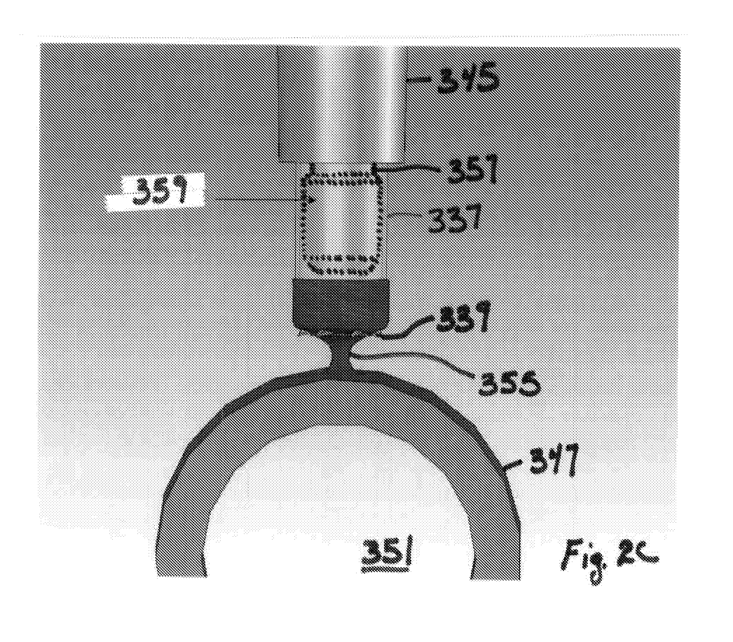

FIGS. 2A-2D illustrate operation of a system for closing a puncture in a vessel wall with an anchoring device and an angled wall grip end.

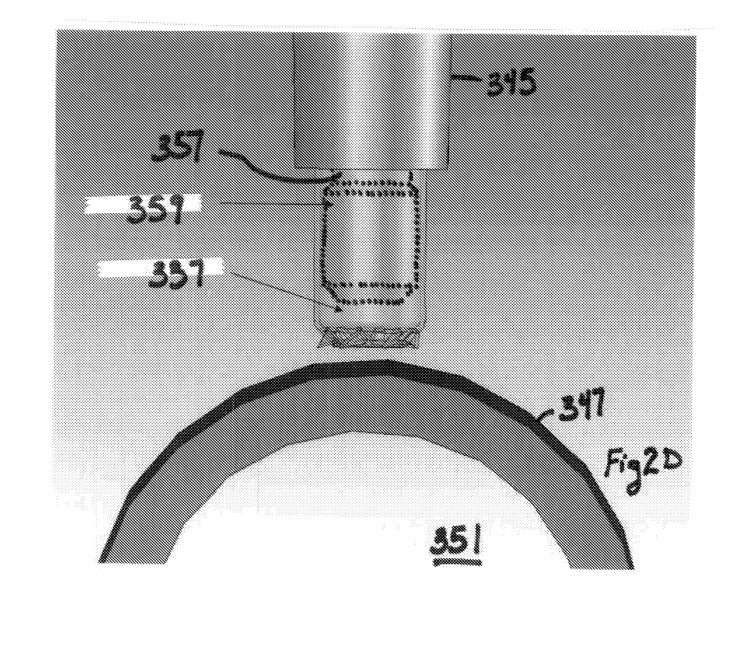

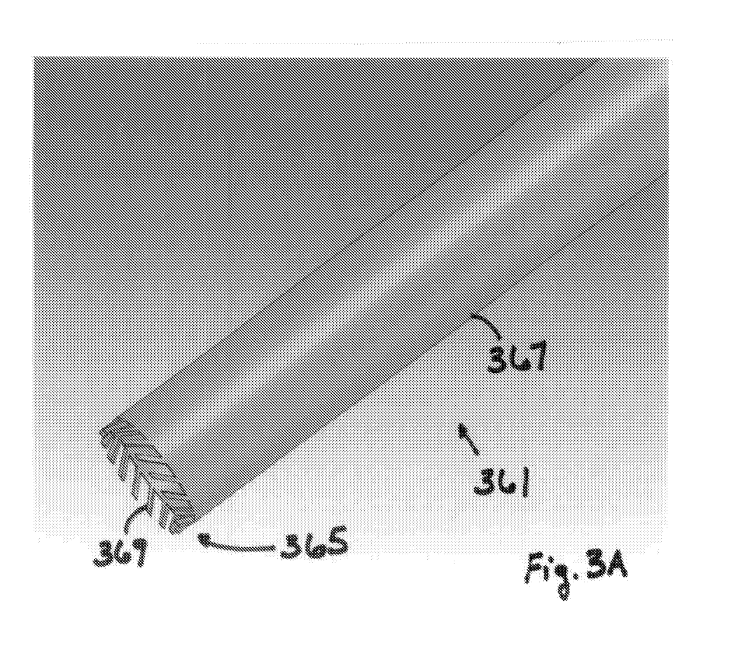

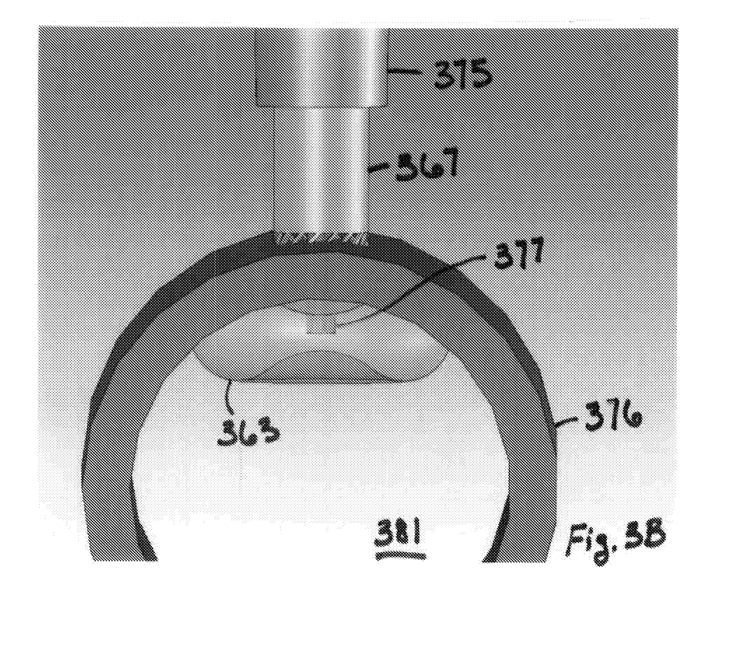

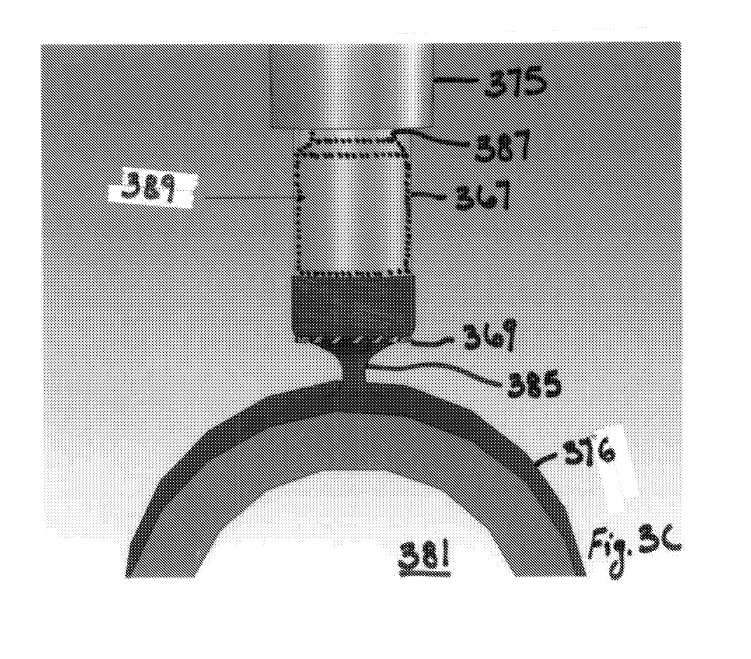

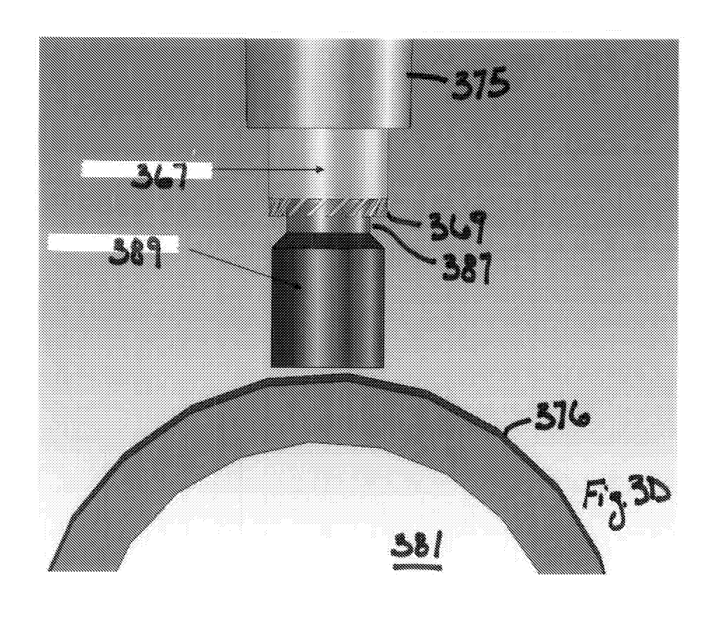

FIGS. 3A-3D illustrate operation of a system for closing a puncture in a vessel wall with an anchoring device and a tip grip.

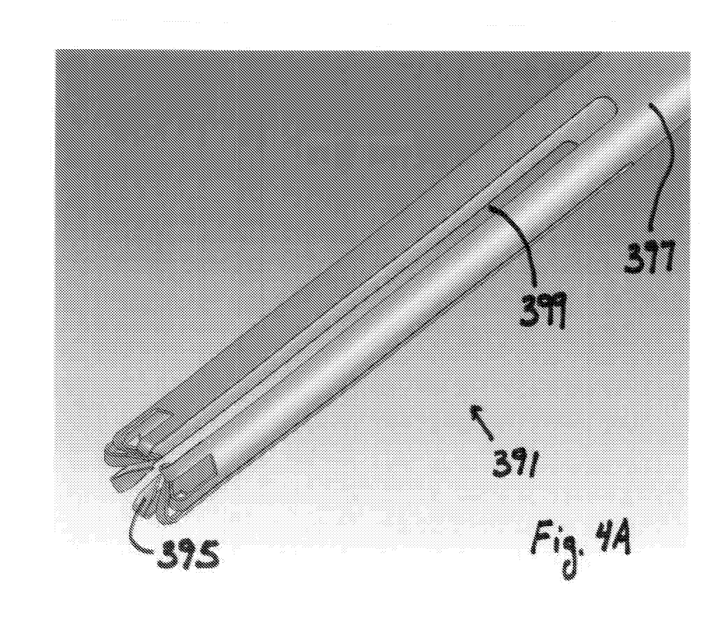

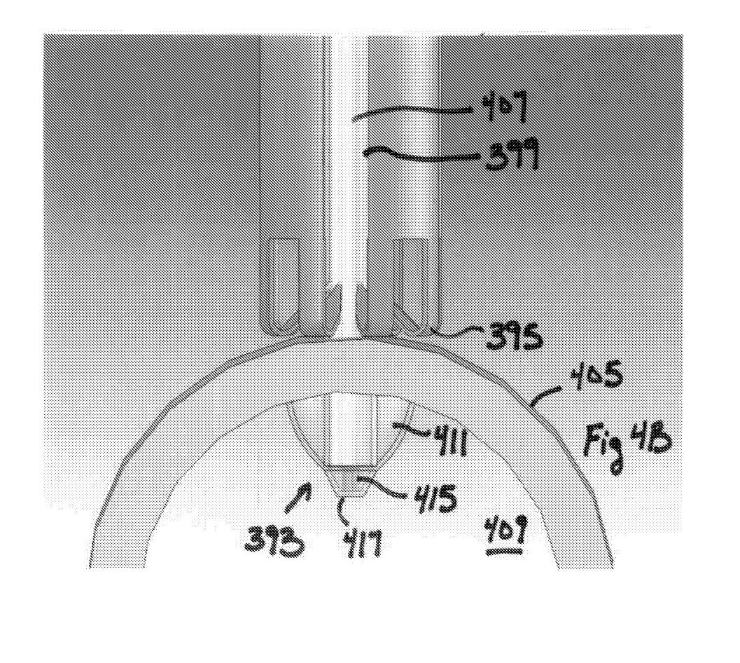

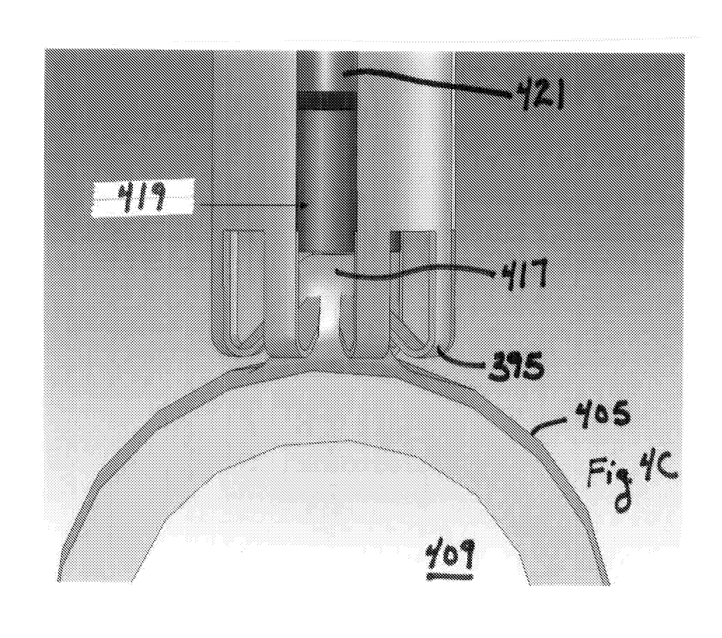

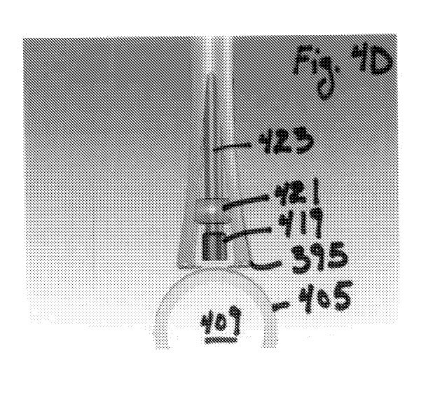

FIGS. 4A-4D illustrate operation of a system for closing a puncture in a vessel wall with an anchoring device and inward facing tip prongs.

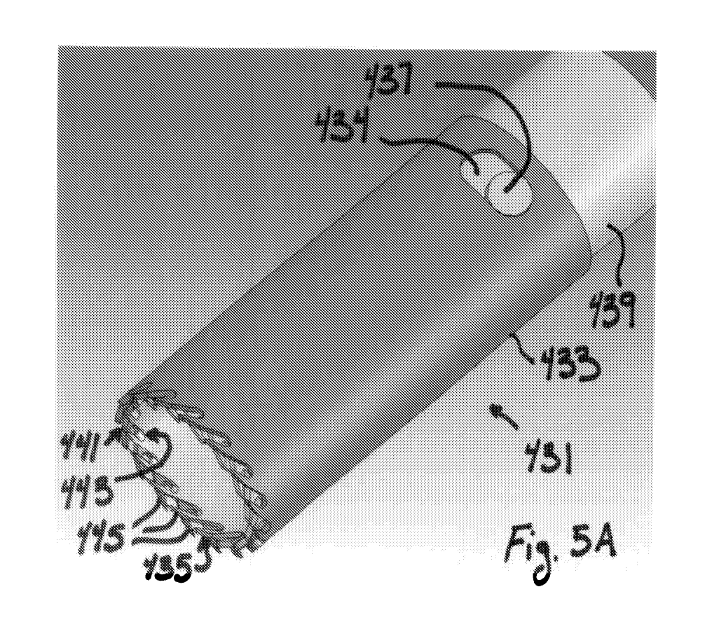

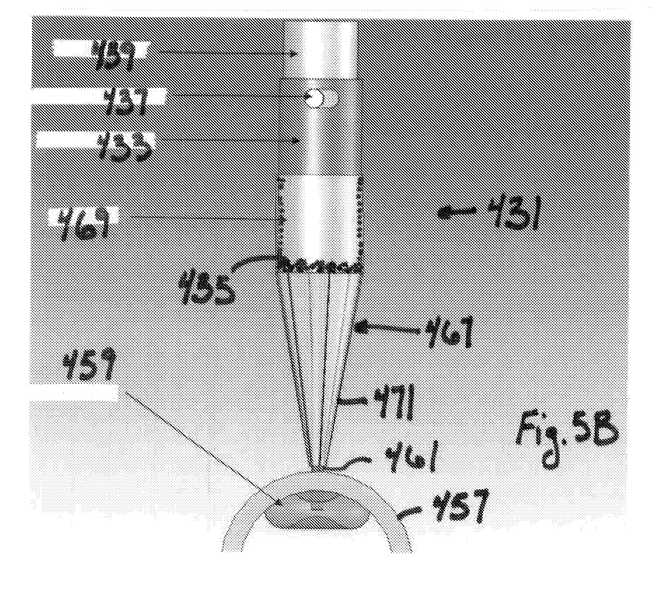

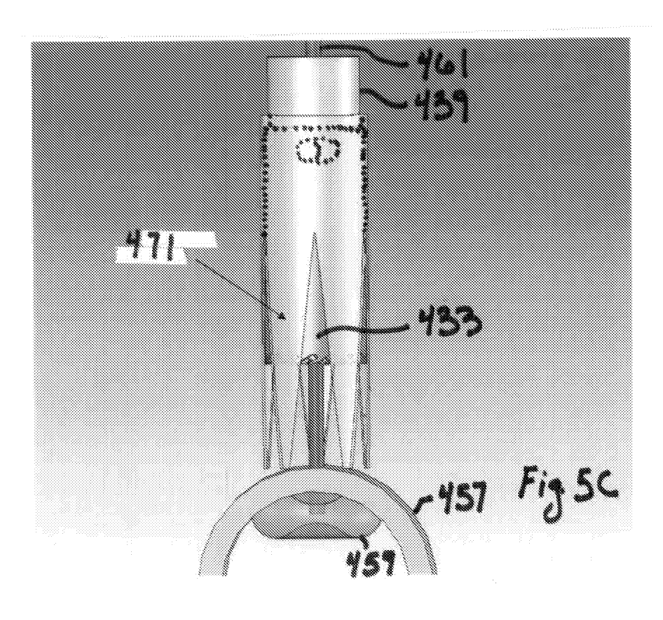

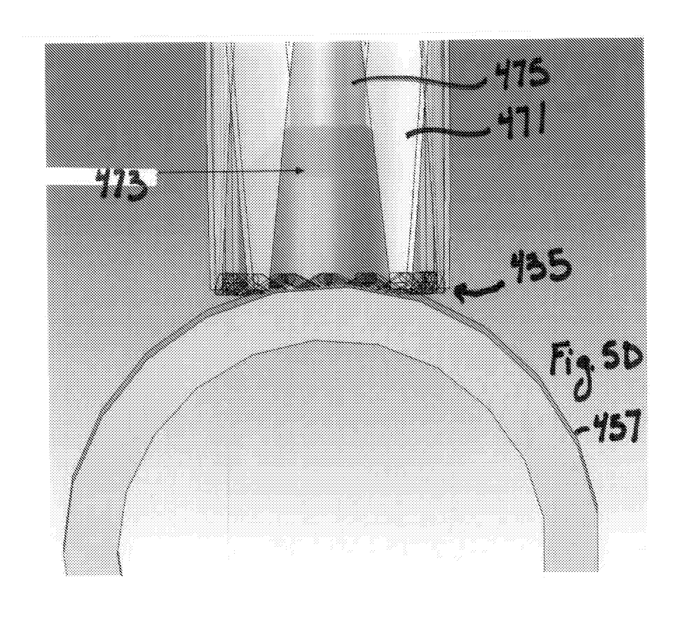

FIGS. 5A-5D illustrate a system for closing a puncture in a vessel wall with a double wall grip.

### DETAILED DESCRIPTION OF THE PREFERRED EMBODIMENTS

Embodiments of the present invention may include apparatus and methods for sealing a puncture in a vessel wall A system for closing a puncture in a vessel wall may have one or more gripping elements and a heating element for sealing a puncture within a vessel wall. An anchoring device may be a balloon, a floating anchor, an expansible cage and/or a wire operated T-anchor. The heating element may be directly in contact with a vessel wall or may be placed in the vicinity of the vessel wall. The various embodiments of the present invention may be used in various combinations depending on a particular application.

Anchoring systems may be used to provide stability during a procedure. Anchoring systems may act as a locating system. Anchoring systems of the present invention may generally be inserted into a vessel through an apparatus. The anchoring systems may then be deployed with a vasculature and withdrawn into contact with an inner surface of a vessel wall. The anchoring systems may be used interchangeably with the various embodiments of the present invention depending on a particular application.

Heating elements may be used to close a puncture within a vessel wall. Heating elements of the present invention are preferably direct contact conduction heating elements. Other types of heating elements, however, such as radio frequency, laser and others, may be used for particular applications. Heating elements may have various shapes for various applications. Flat-ended heating elements may be preferred for applications where the apparatus is inserted perpendicular to a vessel wall. A flat-ended heating element may allow more heating surface into contact with a vessel wall. Dome-shaped heating elements may be preferably used in applications where the apparatus is inserted at various angles in comparison with a vessel wall. Note that a dome-shaped heating element may also be desirable in situations where an apparatus is inserted perpendicular to a vessel wall. Heating elements may be rigid or flexible depending on particular applications. Heating elements may be heated by passing current through a wire or other similar conducting element. Conducting elements may have various configurations to deliver customized heating patterns for particular applications. Various patterns of conducting elements may allow for various treatments.

FIGS. 1A-1F illustrate operation of a system for closing a puncture in a vessel wall 301 with an anchoring device 303 and a wall grip end 305. The system for closing a puncture in a vessel wall 301 may be used with any of the anchoring devices and/or heating elements as described above. The following description is for illustrative purposes only.

FIG. 1A shows an apparatus 301 with a cylindrical shaft 307 and a wall grip end 305. The wall grip end may have one or more protrusions 309 and one or more openings 311. The one or more protrusions 309 may be integrated with or formed from the one or more openings 311. The one or more protrusions 309 may form points or may have sharp edges or corners. FIG. 1B shows an introducer assembly 313 with an introducer shaft 315 passing through a vessel wall 317. The introducer assembly 313 may be used during a procedure. After a procedure, a folded anchor 303 on an anchor shaft 319 may be passed through the introducer shaft 315 and into a vessel lumen 321. The folded anchor 303 may be a balloon or another type of anchor device. The folded anchor 303 may then be inflated to a desired size, as shown in FIG. 1C, and withdrawn into contact with an inner surface of the vessel wall 317 below a puncture. The apparatus 301 may be inserted into the introducer apparatus 313. The apparatus 301 may be covered by a protective tube 323. The apparatus 301 may be lowered into contact with an outer surface of the vessel wall 317 above a puncture as shown in FIG. 1D. Preferably, the protecting tube 323 remains within the introducer shaft 315. The apparatus 301 may be twisted or rotated while the anchor device 303 remains in position as shown in FIG. 1E. The protrusions 309 may catch on the vessel wall 317 during twisting of the apparatus 301. The apparatus 301 may be twisted such that a section 325 of the vessel wall 317 surrounds the anchor shaft 319 and prevents passage of blood. Twisting may be anywhere from one-quarter or less to several rotations of the apparatus 301. The apparatus 301 may be withdrawn slightly from the vessel wall 317 during twisting. The anchor device 303 may be compressed or deflated and retracted through the cylindrical shaft 307. A heating element 329 on a heating element shaft 327 may be lowered through the cylindrical shaft 307 into contact with the twisted section 325 of the vessel wall 317. The heating element 329 may be activated to heat and seal the twisted section 325 of the vessel wall 317. Alternatively, the apparatus 301 itself may be heated to seal the twisted section 325 of the vessel wall 317. The twisted section 325 of the vessel wall 317 may be released by heating or mechanical forces as shown in FIG. 1F. Heat may cause the tissue to pull away from the apparatus 301. Mechanical forces, such as twisting in a reverse direction, may also release the tissue. The apparatus 301 may then be withdrawn from the outer surface of the vessel wall 317. The apparatus 301 may be withdrawn into the introducer shaft 315. The heating element 329 and heating element shaft 327 may be withdrawn simultaneously, before or after the withdrawal of the apparatus 301.

FIGS. 2A-2D illustrate operation of a system for closing a puncture in a vessel wall 331 with an anchoring device 333 and an angled wall grip end 335. The system for closing a puncture in a vessel wall 331 may be used with any of the anchoring devices and/or heating elements as described above. The following description is for illustrative purposes only.

FIG. 2A shows an apparatus 331 with a cylindrical shaft 337 and a wall grip end 335. The wall grip end 335 may have one or more protrusions 339 and one or more openings 341. The one or more protrusions 339 may be integrated with or formed from the one or more openings 341. The one or more protrusions 339 may form points or may have sharp edges or corners. The one or more protrusions 339 and the one or more openings 341 may be disposed on a slanted wall at the end of the cylindrical shaft 337. The slanted wall 314 is preferably slanted inward toward the center of the cylindrical shaft at about a 45 degree angle, but could be any appropriate angle for particular applications. An introducer assembly similar to that used in FIG. 1 may be used with various embodiments of the present invention. For example, an introducer assembly with an introducer shaft 345 may pass through a vessel wall. The introducer assembly may be used during a procedure. After a procedure, a folded anchor 333, similar to that used in FIG. 1, on an anchor shaft 349 may be passed through the introducer shaft 345 and into a vessel lumen 351. The folded anchor may be a balloon or another type of anchor device. The folded anchor may then be inflated to a desired size, and withdrawn into contact with an inner surface of the vessel wall below a puncture. The apparatus 331 may be lowered into contact with an outer surface of a vessel wall 347 above a puncture as shown in FIG. 2B. The apparatus 331 may be twisted or rotated while an anchor device 333 on an anchor shaft 349 remains in position as shown in FIG. 2G The protrusions 339 may catch on the vessel wall 347 during twisting of the apparatus 331. The apparatus 331 may be twisted such that a section 355 of the vessel wall 347 surrounds the anchor shaft 349 and prevents passage of blood. Twisting may be anywhere from one-quarter or less to several rotations of the apparatus 331. The apparatus 331 may be withdrawn slightly from the vessel wall 347 during twisting. The anchor device 333 may be compressed or deflated and retracted through the cylindrical shaft 337. A heating element 359 on a heating element shaft 357 may be lowered through the cylindrical shaft 337 into contact with the twisted section 355 of the vessel wall 347. The heating element 359 may be activated to heat and seal the twisted section 355 of the vessel wall 347. Alternatively, the apparatus 331 itself may be heated to seal the twisted section 355 of the vessel wall 347. The twisted section 355 of the vessel wall 347 may be released by heating or mechanical forces as shown in FIG. 2D. Heat may cause the tissue to pull away from the apparatus 331. Mechanical forces, such as twisting in a reverse direction, may also release the tissue. The apparatus 331 may then be withdrawn from the outer surface of the vessel wall 347. The apparatus 331 may be withdrawn into the introducer shaft 345. The heating element 359 and heating element shaft 357 may be withdrawn simultaneously, before or after the withdrawal of the apparatus 331.

FIGS. 3A-3D illustrate operation of a system for closing a puncture in a vessel wall 361 with an anchoring device 363 and a tip grip 365. The system for closing a puncture in a vessel wall 361 may be used with any of the anchoring devices and/or heating elements as described above. The following description is for illustrative purposes only.

FIG. 3A shows an apparatus 361 with a cylindrical shaft 367 and a wall grip end 365. The wall grip end 365 may have one or more protrusions 369. The one or more protrusions 369 may be serrated teeth extending from the wall grip end 365. The one or more protrusions 339 may form points or may have sharp edges or corners and/or may be disposed at an angle relative to the cylindrical shaft 367. An introducer assembly similar to that used in FIG. 1 may be used with various embodiments of the present invention. For example, an introducer assembly with an introducer shaft 375 may pass through a vessel wall. The introducer assembly may be used during a procedure. After a procedure, a folded anchor 363, similar to that used in FIG. 1, on an anchor shaft 377 may be passed through the introducer shaft and into a vessel lumen. The folded anchor may be a balloon or another type of anchor device. The folded anchor may then be inflated to a desired size, and withdrawn into contact with an inner surface of the vessel wall below a puncture. The apparatus 361 may be lowered into contact with an outer surface of the vessel wall 376 above a puncture as shown in FIG. 3B. The apparatus 361 may be twisted or rotated while the anchor device 363 remains in position as shown in FIG. 3C The protrusions 369 may catch on the vessel wall 376 during twisting of the apparatus 361. The apparatus 361 may be twisted such that a section 385 of the vessel wall 376 surrounds the anchor shaft 379 and prevents passage of blood. Twisting may be anywhere from one-quarter or less to several rotations of the apparatus 361. The apparatus 361 may be withdrawn slightly from the vessel wall 376 during twisting. The anchor device 363 may be compressed or deflated and retracted through the cylindrical shaft 367. A heating element 389 on a heating element shaft 387 may be lowered through the cylindrical shaft 367 into contact with the twisted section 385 of the vessel wall 376. The heating element 389 may be activated to heat and seal the twisted section 385 of the vessel wall 376 as shown in FIG. 3G Alternatively, the apparatus 361 itself may be heated to seal the twisted section 385 of the vessel wall 376. The twisted section 385 of the vessel wall 376 may be released by heating or mechanical forces as shown in FIG. 3D. Heat may cause the tissue to pull away from the apparatus 361. Mechanical forces, such as twisting in a reverse direction, may also release the tissue. The apparatus 361 may then be withdrawn from the outer surface of the vessel wall 376. The apparatus 361 may be withdrawn into the introducer shaft 375. The heating element 389 and heating element shaft 387 may be withdrawn simultaneously, before or after the withdrawal of the apparatus 361.

FIGS. 4A-4D illustrate operation of a system for closing a puncture in a vessel wall 391 with an anchoring device 393 and inward facing tip prongs 395. The system for closing a puncture in a vessel wall 391 may be used with any of the anchoring devices and/or heating elements as described above. The following description is for illustrative purposes only.

FIG. 4A shows an apparatus 391 with a cylindrical shaft 397, one or more extended vertical grooves 399 running along a vertical axis of the apparatus 391, and one or more inward facing tip prongs 395 at a distal end of the apparatus 391. The inward facing tip prongs 395 may have pointed tips or sharp edges for gripping tissue. The prongs 395 may be grouped into sets of two or more between each groove 399. An introducer assembly similar to that used in FIG. 1 may be used with various embodiments of the present invention. For example, an introducer assembly with an introducer shaft may pass through a vessel wall. The introducer assembly may be used during a procedure. After a procedure, a folded anchor 393, similar to that used in FIG. 1, on an anchor shaft 407 may be passed through the introducer shaft and into a vessel lumen. The folded anchor may be a balloon or another type of anchor device. The folded anchor may then be inflated to a desired size, and withdrawn into contact with an inner surface of the vessel wall below a puncture. Preferably, the anchor 393 is a cup-shaped element 411 as shown in FIG. 4B. The cup-shaped element may be inserted into the vessel lumen 409 and withdrawn into contact with an inner surface of the vessel wall 405. The introducer shaft may be withdrawn. The apparatus 391 may be inserted into the introducer apparatus. The apparatus 391 may be lowered until the prongs 393 contact with an outer surface of the vessel wall 405 above a puncture. The anchor device 393 may be flipped for an inverted cup-shape position. The inversion may be accomplished by operating an inversion shaft 415 connected to an inversion end 417. The inversion shaft 415 may be coaxially movable relative to the anchor shaft 407. The inverted anchor member 393 may then be withdrawn towards the apparatus 391. The withdrawal of the inverted anchor member 393 may pull part 417 of the vessel wall 405 with the anchor member 393. The inverted anchor member 393 may be completely withdrawn from the patient through the apparatus 391. The movement of the inverted anchor member 393 may cause the captured tissue 417 to catch on the prong tip ends 393 as shown in FIG. 4C. A heating element 419 on a distal end of a heating element shaft 421 may be lowered through the apparatus 391 into contact with the captured tissue 417. The heating element 419 may be activated to heat and seal a puncture in the vessel wall 405 at the captured tissue 417. A tip expanding mechanism 423 may be activated to separate the prongs 393 holding the captured tissue 417 as shown in FIG. 4D. The tip expanding mechanism 423 may be a balloon or another expandable device. The tip expanding mechanism 423 may be located on the heating element shaft 421 or on a separate coaxial shaft (not shown). Preferably, the tip expanding mechanism 423 separates the prongs 393 far enough to allow release of the captured tissue 417. Once the captured tissue 417 is released, the tip expanding mechanism 423 may be compressed or deflated to allow the prongs 393 to return to an initial configuration. The heating element 419, heating element shaft 421 and the tip expanding mechanism 423 may be withdrawn through the apparatus 391. The apparatus 391 may then be withdrawn from the patient. Alternatively, the apparatus 391, heating element 419, heating element shaft 421 and the tip expanding mechanism 423 may be withdrawn from the patient simultaneously.

FIGS. 5A-5D illustrate a system for closing a puncture in a vessel wall 431 with a double wall grip 435. The system for closing a puncture in a vessel wall 431 may be used with any of the anchoring devices and/or heating elements as described above. The following description is for illustrative purposes only.

FIG. 5A shows a system for closing a puncture in a vessel wall 431 with an external tube 433, double wall grip end 435, twist control pin 437 and an internal tube 439. The wall grip end 435 may have one or more rows 441, 443 of protrusions 445. The one or more rows 441, 443 of protrusions 445 may be configured such that protrusions on alternate rows 441, 443 may face in opposite directions. The protrusions 445 may form points or may have sharp edges or corners. An introducer assembly similar to that used in FIG. 1 may be used with various embodiments of the present invention. For example, an introducer assembly with an introducer shaft may pass through a vessel wall. The introducer assembly may be used during a procedure. After a procedure, a folded anchor 459, similar to that used in FIG. 1, on an anchor shaft 461 may be passed through the introducer shaft and into a vessel lumen. The folded anchor may be a balloon or another type of anchor device. The folded anchor may then be inflated to a desired size, and withdrawn into contact with an inner surface of the vessel wall below a puncture. The introducer apparatus may be withdrawn before insertion of the system for closing a puncture in a vessel wall 431. Alternatively, the system for closing a puncture in a vessel wall 431 may be inserted into the introducer apparatus. The system for closing a puncture in a vessel wall 431 mayor may not be covered by a protective tube (not shown). The system for closing a puncture in a vessel wall 431 may be lowered over the anchor shaft 461 to a predetermined distance above the vessel wall 457, as shown in FIG. 5B, or into contact with an outer surface of the vessel wall 457 above a puncture. A dilator 467 may cover a distal end of the system for closing a puncture in a vessel wall 431. The dilator 467 may have a roughly cylindrical upper end 469 surrounding the external tube 433 and an expansible section 471. Alternative configurations of the dilator 467 are possible depending on particular uses. The apparatus 431 may be pushed toward the vessel wall 457 such that the expansible section 471 of the dilator 467 opens outward. The expansible section 471 may include one or more sections hinged below the upper end 469 of the dilator 467. As the dilator 467 opens from the force of the system for closing a puncture in a vessel wall 431, as shown in FIG. 5C, the system for closing a puncture in a vessel wall 431 may continue moving toward the vessel wall 457 until a distal end of the system for closing a puncture in a vessel wall 431 contacts the vessel wall 457. The double wall grip end 435 of the apparatus 431 may contact the vessel wall 457 as shown in FIG. 5D. The internal tube 439 or another portion of the apparatus 431 may be twisted or rotated against the external tube 433 to trap a portion of the vessel wall 457 with minimal deformation of the vessel wall 457. The twist control pin 437 may prevent over-rotation of the internal tube 439. The twist control pin 437 may allow relative rotation for a predetermined distance by sliding within an opening 434 on the external tube 433. The protrusions 445 may catch on the vessel wall 457 during twisting of the system for closing a puncture in a vessel wall 431. The anchor device 459 may be compressed or deflated and retracted through the system for closing a puncture in a vessel wall 431. A heating element 473 on a heating element shaft 475 may be lowered through the external tube 433 into contact with the twisted section of the vessel wall 457. The heating element 473 may be activated to heat and seal the twisted section of the vessel wall 457. Alternatively, the apparatus 431 itself may be heated to seal the twisted section of the vessel wall 457. The twisted section of the vessel wall 457 may be released by heating or mechanical forces. Heat may cause the tissue to pull away from the system for closing a puncture in a vessel wall 431. Mechanical forces, such as twisting in a reverse direction, may also release the tissue. The apparatus 431 may then be withdrawn from the outer surface of the vessel wall 457. The apparatus 431 may be withdrawn into the introducer shaft 455 if an introducer is used. The heating element 473 and heating element shaft 475 may be withdrawn simultaneously, before or after the withdrawal of the system for closing a puncture in a vessel wall 431.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. A system for closing a puncture in a vessel wall comprising:
an anchor shaft,
an anchoring device on a distal end of an anchor shaft wherein the anchoring device is deployed within a vessel lumen and withdrawn into contact with an inner surface of a vessel wall,
a gripping shaft for passing over the anchor shaft,
a gripping tip on a distal end of the gripping shaft for contacting an outer surface of the vessel wall and capturing tissue during rotation of the shaft, and
a heating element for passing through the gripping shaft into contact with the captured tissue.

2. The system of claim 1, further comprising an introducer wherein the gripping shaft passes through the introducer.

3. The system of claim 1, further comprising a sheath for covering the gripping shaft during passing over the anchor shaft.

4. The system of claim 1, wherein the anchoring device is selected from the group consisting of a balloon, a T-anchor, an expansible cage, a floating anchor, and combinations thereof.

5. The system of claim 1, wherein the anchoring device is withdrawn through the shaft prior to activation of the heating element.

6. The system of claim 1, wherein the gripping tip comprises extensions for gripping tissue.

7. The system of claim 6, wherein the extensions are on an inward-sloped wall at the distal end of the gripping shaft.

8. A method of operating a system for closing a puncture in a vessel wall comprising:
providing a system for closing a puncture in a vessel wall comprising:
an anchor shaft,
an anchoring device on a distal end of an anchor shaft, and
a gripping shaft for passing over the anchor shaft,
a gripping tip on a distal end of the gripping shaft, and
a heating element,
inserting the distal end of the anchor shaft into a vessel lumen,
deploying the anchoring device within the vessel lumen,
withdrawing the anchoring device into contact with an inside surface of a vessel wall,
inserting the gripping shaft near the vessel wall,
contacting the gripping tip with an outside surface of the vessel wall opposite the anchoring device,
rotating the gripping shaft for capturing tissue,
withdrawing the anchoring device through the gripping shaft,
contacting the captured tissue with the heating element, and
activating the heating element.

9. The method of claim 8, further comprising releasing the captured tissue after activating the heating element.

10. The method of claim 9, wherein the releasing is performed by rotating the gripping shaft in an opposite direction from the initial rotation.

11. The method of claim 8, wherein the gripping shaft is inserted through an introducer.

12. The method of claim 8, wherein the gripping shaft is covered by a protective sheath during insertion.

13. The method of claim 8, wherein the anchoring device is selected from the group consisting of a balloon, a T-anchor, an expansible cage, a floating anchor, and combinations thereof.

14. The method of claim 8, wherein the gripping tip comprises extensions for gripping tissue.

15. The method of claim 14, wherein the extensions are on an inward-sloped wall at the distal end of the gripping shaft.

16. A system for closing a puncture in a vessel wall comprising:
an anchor shaft,
an anchoring device on a distal end of the anchor shaft wherein the anchoring device is deployed within a vessel lumen and withdrawn into contact with an inner surface of a vessel wall,
a second shaft,
one or more prongs on a distal end of the second shaft for capturing tissue at an outer surface of the vessel wall opposite the anchor device, and
a heating element within the second shaft for contacting the captured tissue.

17. The system of claim 16, further comprising an expansion device within the second shaft for releasing the captured tissue.

18. The system of claim 17, wherein the expansion device is a balloon.

19. The system of claim 16, wherein the one or more prongs are inward facing.

20. The system of claim 16, wherein the one or more prongs are disposed in two or more concentric rows.

21. The system of claim 20, wherein the one or more prongs are angled in opposite directions in adjacent concentric rows.

22. The system of claim 16, wherein the anchoring device is selected from the group consisting of a balloon, a T-anchor, an expansible cage, a floating anchor, and combinations thereof.

23. The system of claim 16, wherein the anchoring device is an inverting cup and deployed in a first position, inverted, and withdrawn to capture tissue on the one or more prongs.

24. The system of claim 16, wherein the second shaft further comprises a proximate internal tube and a distal external tube.

25. The system of claim 24, wherein the internal tube rotates relative to the external tube.

26. The system of claim 25, further comprising twist control pin for preventing over-rotation of the internal tube.

27. The system of claim 24, further comprising a dilator with an expansible section, wherein the dilator at least partially covers the distal external tube.

28. The system of claim 27, wherein the expansible section opens upon movement of the external tube toward a vessel wall.

29. A method of operating a system for closing a puncture in a vessel wall comprising:
providing a system for closing a puncture in a vessel wall comprising:
an anchor shaft,
an anchoring device on a distal end of the anchor shaft,
a second shaft,
one or more prongs on a distal end of the second shaft, and
a heating element within the second shaft,
inserting the distal end of the anchor shaft into a vessel lumen,
deploying the anchoring device within the vessel lumen,
withdrawing the anchoring device into contact with an inner surface of a vessel wall,
inserting the second shaft over the anchor shaft proximate to an outer surface of the vessel wall opposite the anchoring device,
capturing tissue on the one or more prongs, and
applying the heating element to the captured tissue.

30. The method of claim 29, further comprising withdrawing the anchoring device through the second shaft to capture the tissue on the one or more prongs.

31. The method of claim 30, wherein the anchoring device is an inverting cup that is inverted prior to withdrawal through the shaft.

32. The method of claim 29, further comprising deploying an expansion device within the second shaft to release the captured tissue and undeploying the expansion device.

33. The method of claim 32, wherein the expansion device is a balloon.

34. The method of claim 29, wherein the anchoring device is selected from the group consisting of a balloon, a T-anchor, an expansible cage, a floating anchor, and combinations thereof.

35. The method of claim 29, wherein the one or more prongs are inward facing.

36. The method of claim 29, further comprising withdrawing the anchoring device through the second shaft after capturing the tissue on the one or more prongs by rotating the one or more prongs.

37. The method of claim 36, wherein the shaft comprises a proximate inner tube rotatable relative to a distal external tube.

38. The method of claim 29, further comprising providing a dilator with an expansible section and opening the expansible section by moving the external tube toward a vessel wall.

39. The method of claim 29, wherein the one or more prongs are disposed in two or more concentric rows.

40. The method of claim 39, wherein the one or more prongs are angled in opposite directions in adjacent concentric rows.
